(19) United States
(12) Patent Application Publication
(10) Pub. No.: US 2021/0236769 A1
(43) Pub. Date: Aug. 5, 2021

(54) BALLOON CATHETER

(71) Applicant: LIPAC ONCOLOGY LLC, Menlo Park, CA (US)

(72) Inventor: Michael G. OEFELEIN, Tustin, CA (US)

(21) Appl. No.: 17/051,981

(22) PCT Filed: May 6, 2019
(86) PCT No.: PCT/US2019/030833

§ 371 Date: Oct. 30, 2020

Related U.S. Application Data
(60) Provisional application No. 62/666,848, filed on May 4, 2018.
(60) Provisional application No. 62/680,172, filed on Jun. 4, 2018.

(51) Int. Cl. A61M 25/00; A61M 25/10; A61K 47/24; A61K 31/337; A61K 9/127

(52) U.S. Cl. CPC A61M 25/0017; A61M 25/10186; A61K 47/24; A61M 2025/1052; A61K 9/127; A61M 2025/1056; A61K 31/337

(57) ABSTRACT

The invention locally delivers a chemotherapeutic or a radiographic contrast agent to an upper tract urothelial carcinoma (UTUC). The invention includes a balloon catheter with a working channel and a balloon placed into the ureter/renal pelvis via retrograde or antegrade ureteral access. The balloon is inflated to temporarily obstruct the ureter, and a formulation with a chemotherapeutic agent is infused into the working channel of the catheter and allowed to dwell in the ureter/renal pelvis for a time sufficient to allow the formulation to adhere to and penetrate the urothelial wall. The formulations can be liposomal or non-liposomal formulations. At least portion of the infused chemotherapeutic-agent formulation adheres to the urothelial wall while it is instilled and dwells in the ureter/renal pelvis. The methods of the invention can be performed as an adjuvant therapy to other methods of treating UTUC, including ureteroscopic ablation or resection of the tumor.

### CROSS REFERENCE TO RELATED APPLICATION

This application claims the benefit of U.S. Provisional Application No. 62/666,848, filed on May 4, 2018 and U.S. Provisional Application No. 62/680,172 filed on Jun. 4, 2018. This application incorporates by reference the entire contents of U.S. Provisional Application No. 62/666,848, filed on May 4, 2018, and U.S. Provisional Application No. 62/680,172 filed on Jun. 4, 2018.

### TECHNICAL FIELD

The invention relates to an apparatus and methods for locally delivering a chemotherapeutic agent to an upper tract urothelial carcinoma (UTUC). The invention includes a catheter to affect local delivery of liposomal and other therapeutic formulations into the ureter and/or renal pelvis via retrograde or antegrade catheter access.

### BACKGROUND

The urothelium is the layer of tissue that lines the bladder and the upper urinary tract. The urothelium swells and shrinks to push urine through the urinary tract. Because the urothelium is in direct contact with the urine, this lining is exposed to chemicals (including carcinogens) that are filtered out of the blood by the kidneys. These chemicals can cause cells to change and grow out of control as cancer.

Upper tract urothelial carcinoma (UTUC) is a relatively uncommon type of cancer, accounting for only 5-10% of all urothelial cancers. The estimated annual incidence of UTUC in Western countries is about two cases per 100,000 inhabitants (or 6000 cases/year). UTUCs have a peak incidence in people aged 70-90 years, and they are three times more common in men. In 17% of cases of the disease, concurrent bladder cancer is present. Recurrence in the bladder occurs in 22-47% of UTUC patients, compared with 2-6% in the contralateral upper tract. Sixty percent of UTUCs are invasive at diagnosis, compared with 15-25% of bladder tumors.

Treatment of UTUC depends upon the tumor grade (how aggressive), tumor size, patient age, medical history, overall health, and the patient's kidney collecting system anatomy. Treatment options include surgery, radiation, and chemotherapy.

Treatment of UTUC in high-risk patients may require radical nephroureterectomy (RNU). On the other hand, low-risk cases can involve conservative management, which can be considered when the contralateral kidney is functional. Kidney-sparing surgery (KSS) for low-risk UTUC allows avoiding the morbidity associated with open radical surgery, without compromising oncological outcomes and kidney function. In addition, conservative management can also be considered in all imperative cases (i.e., in patients with renal insufficiency or solitary functional kidney). The most common KSS approaches include ureteroscopic retrograde tumor ablation, percutaneous anterograde tumor ablation, or segmental ureterectomy.

Endoscopic ablation of UTUC using ureteroscopy can be considered in highly selected cases and in the following situations: laser generator and alligator type endoscopic forceps are available for biopsies; flexible and rigid ureteroscope; when the patient is informed of the need for closer, more stringent, surveillance; and when a complete tumor resection is strongly advocated. However, endoscopic access to remote anatomical regions of the renal pelvis may limit the ability to surgically ablate or biopsy. In addition, with pure endoscopic management, there is a risk of under staging and under grading the tumor. Furthermore, recurrence rates for renal pelvic and ureteral tumors treated with ureteroscopy are high, 33-35% and 31-32%, respectively.

To combat tumor recurrence, surgical management of UTUC can be accompanied by postoperative instillation therapy using antitumor chemotherapeutic agents. In UTUC, intra-luminal instillation of MMC has been reported following KSS with promising results. However, because of the difficulties in uretero-pelvic access and achieving adequate drug dwell time, conventional topical chemotherapy administration after endoscopic management has a sub-optimal effect on disease recurrence. Conventional systems are unable to provide intra-luminal persistence and a controlled drug release from the delivery device. It is often necessary to control and/or lengthen the time period over which the drug is released. For example, it may be advantageous to lengthen the release time from seconds to minutes, or from minutes to hours, days, or even weeks. Further, it is often desired to control the release rate of the drug over prolonged periods of time.

The continued recurrence of urothelial tumors even after surgical removal and local postoperative chemotherapy highlights the need for additional treatment measures for UTUC patients. Local administration of drugs poses special challenges in cases involving the walls of ducts and vessels, since these organs serve as transport systems. Inter-luminal delivery into the upper urinary tract poses a unique challenge given the aqueous environment of the ureteral contents, expulsive peristalsis, and the difficulties in penetrating the urothelial barrier.

Prior balloon catheters do not provide optimal drug delivery capabilities and suffer deficiencies with regard to the balloon size and shape and do not include an effective means to check and regulate the pressure in the ureter and/or renal pelvis to safely deliver intraluminal chemotherapeutic agents.

### SUMMARY

The invention provides endoscopic methods of treating UTUC that involve instillation therapy using antitumor chemotherapeutic agents. The invention includes methods of treating UTUC where the dwell time of the chemotherapeutic agent is prolonged, thus allowing increased exposure (and potentially penetration) of the urothelial carcinoma to chemotherapeutic agents. The invention provides devices and methods that use stable liposomal formulations of a chemotherapeutic agent, where the chemotherapeutic agent does not precipitate in the urine pH (acidic), and where the formulations allow for the agent to at least partially adhere to the urothelial wall of the ureter and/or renal pelvis affected by UTUC. Such methods can minimize side effects of the therapy while increasing or maintaining its efficacy.

The invention further includes devices and methods to treat upper tract urothelial carcinoma. One method according to the invention for locally delivering a chemotherapeutic agent to an upper tract urothelial carcinoma (UTUC) places a balloon catheter with a working channel, via retrograde or antegrade ureteral access, into the ureter and/or renal pelvis. The catheter balloon is inflated such that the ureter is temporarily obstructed, and a liquid liposomal formulation comprising a chemotherapeutic agent is instilled into the working channel of the catheter. This allows the instilled liposomal formulation to dwell in the ureter and/or renal pelvis for a time sufficient to allow at least a portion of the liposomal formulation to adhere to (and potentially partially penetrate) the urothelial wall.

One example of the invention includes a catheter for administering a drug to a target site in a body lumen. One example of the invention includes delivering a chemotherapeutic agent via the catheter to treat a UTUC. The catheter itself includes a guidewire/drainage/instillation (GDI) lumen and a balloon inflation (BI) lumen that are combined into a lumen shaft at a Y-body Luer hub. The lumen shaft includes both the guidewire/drainage/instillation (GDI) lumen and a balloon inflation (BI) lumen arranged in the lumen shaft. The balloon inflation (BI) lumen provides a fluid communication path from a balloon inflation solution to an inflatable catheter balloon. The inflated balloon stops the flow of contrast material out of the intrarenal collecting system, and the guidewire/drainage/instillation (GDI) lumen provides a fluid communication path from a proximal end of the guidewire/drainage/instillation (GDI) lumen to a distal tip of the lumen shaft and can be used to drain fluid in a body lumen, such as a ureter, for example. At the proximal end of the guidewire/drainage/installation (GDI) lumen, the catheter includes a pressure check valve that regulates the pressure in a luminal organ. When pressure in the luminal organ (and therefore in the guidewire/drainage/instillation (GDI) lumen reaches a predetermined pressure, the check valve opens to release the pressure. Additionally, the balloon includes marker bands attached to an outer surface of the lumen shaft, to radiologically confirm catheter position. Another example embodiment of the invention includes a catheter for retrograde pyelography with a check valve to minimize pyelo-venous backflow and anatomical distortion of the upper urinary tract. Contrast (and other) material can be drained from the intrarenal collecting system via the check valve to minimize pyelo-venous backflow.

One example embodiment of the invention includes a catheter having a proximal end and a distal end. The catheter includes a balloon inflation lumen in fluid communication with a balloon infusion port in the proximal end of the catheter and also in fluid communication with an expandable occlusion balloon in the distal end of the catheter. The catheter also includes a guidewire/drainage/instillation lumen in fluid communication with a guidewire/drainage/instillation port in the proximal end of the catheter and in fluid communication with at least one drainage hole in the distal end of the catheter. The guidewire/drainage/instillation lumen passes urine from the at least one drainage hole to the guidewire/drainage/instillation port.

The catheter also includes a guidewire entrance hole in fluid communication with the guidewire/drainage/instillation lumen and located in the distal end of the catheter. The guidewire entrance hole is used to load and advance the catheter over a guidewire with the guidewire passing through the guidewire entrance hole, the guidewire/drainage/instillation lumen, and the guidewire/drainage/instillation port.

The catheter also includes a pressure relief valve disposed along the guidewire/drainage/instillation lumen between the guidewire/drainage/instillation port and the expandable occlusion balloon and configured to release luminal fluid into the guidewire/drainage/instillation lumen when a predetermined ureteral pressure is exceeded. Additionally, the expandable occlusion balloon is in fluid communication with the balloon inflation lumen and is disposed at the distal end of the catheter. The expandable occlusion balloon is configured to withstand a force greater than the predetermined ureteral release pressure of the valve. In one example embodiment of the invention, the expandable occlusion balloon is configured to be expanded within a ureter of a patient. In one example embodiment of the invention, the expandable occlusion balloon is configured to be expanded within a ureteral lumen of a patient. In one example embodiment of the invention, the expandable occlusion balloon is an occlusion balloon. In one example embodiment of the invention, the expandable occlusion balloon is a dilation balloon.

In one example embodiment of the invention, at least one of the balloon infusion port and the guidewire/drainage/instillation port is detachable from the catheter.

One example embodiment of the invention includes a method for treating upper tract urothelial carcinoma (UTUC) in a subject in need of treatment. The method includes delivering a chemotherapeutic agent to the ureter or renal pelvis of the subject. The method includes placing a catheter in accordance with the invention into the ureter and expanding the occlusion balloon of the catheter such that the ureter or renal pelvis is temporarily obstructed. The method also includes loading a formulation comprising a therapeutic dose of a chemotherapeutic drug into the guidewire/drainage/instillation lumen of the catheter and instilling the formulation into the ureter or renal pelvis. The method further includes allowing the instilled formulation to dwell in the ureter or renal pelvis for a time sufficient to allow at least a portion of the formulation to adhere to and partially penetrate the urothelial wall.

In one embodiment of the invention, the chemotherapeutic agent is at least one of a taxane class drug, a platinum-based drug, 5-fluorouracil, mitomycin C, gemcitabine, epirubacin, thiotepa, or a combination thereof. In one embodiment of the invention, the formulation is a liposomal formulation of a chemotherapeutic agent. In one embodiment of the invention, the liposomal formulation includes a taxane class drug or a platinum-based drug and at least one phospholipid component. In one embodiment of the invention at least one phospholipid component is a combination of dimyristoyl phosphatidylcholine (DMPC) and dimyristoyl phosphatidyl glycerol sodium (DMPG). In one embodiment of the invention, the liposomal formulation includes paclitaxel, DMPC and DMPG. In one embodiment of the invention, the liposomal formulation includes paclitaxel, DMPC and DMPG in respective weight/weight ratios of 1:(1-10):(1-10).

In one embodiment of the invention, the instilled formulation is allowed to dwell in the ureter or renal pelvis for at least 1 to 120 minutes.

One example embodiment of the invention includes a method for visualizing a ureter or renal pelvis of a subject. The method includes placing a catheter in accordance with the invention into the ureter; expanding the occlusion balloon of the catheter such that the ureter or renal pelvis is temporarily obstructed; loading a composition comprising a radiographic contrast agent into the guidewire/drainage/instillation lumen of the catheter; instilling the composition comprising the radiographic contrast agent into the ureter or renal pelvis; and allowing the instilled composition to dwell in the ureter or renal pelvis for a time sufficient to allow to allow visualization of the ureter or renal pelvis with fluoroscopy or radiography, while minimizing pyelo-venous backflow exposure to the patient.

### BRIEF DESCRIPTION OF THE DRAWINGS

FIGS. 1A and 1B illustrate an exemplary catheter in accordance with the invention.

FIG. 2 depicts delivery of a liposomal formulation of a chemotherapeutic formulation into the ureter and renal pelvis via retrograde access. Line 1 depicts a ureteroscope.

FIG. 3 shows a perspective view of an exemplary catheter of the invention.

FIGS. 4A and 4B show a top view and a side view, respectively, of an exemplary catheter of the invention.

FIG. 5 shows exemplary valves used in a catheter of the invention.

### DETAILED DESCRIPTION

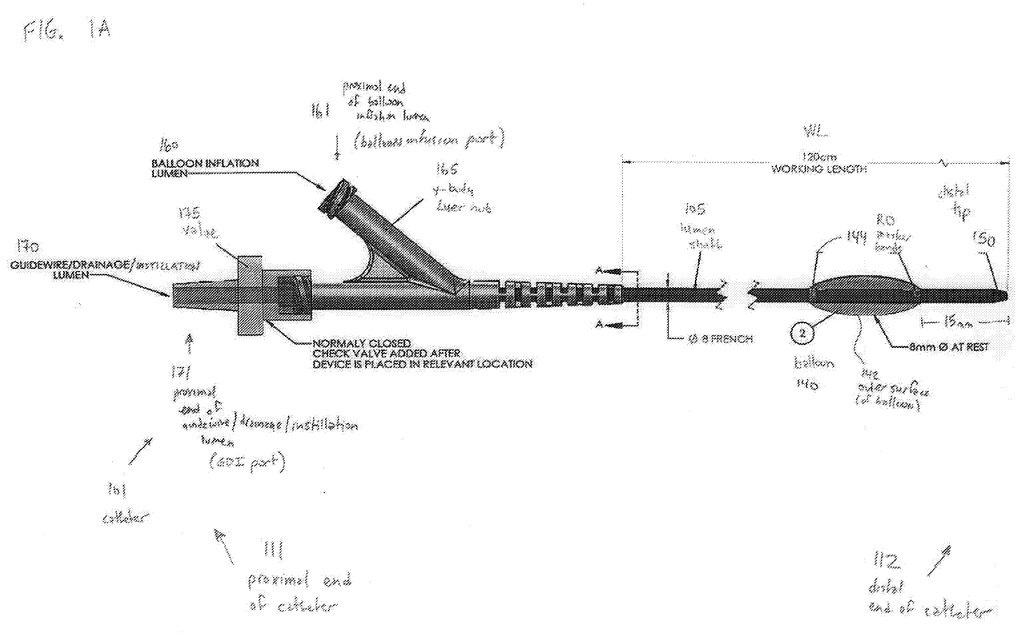

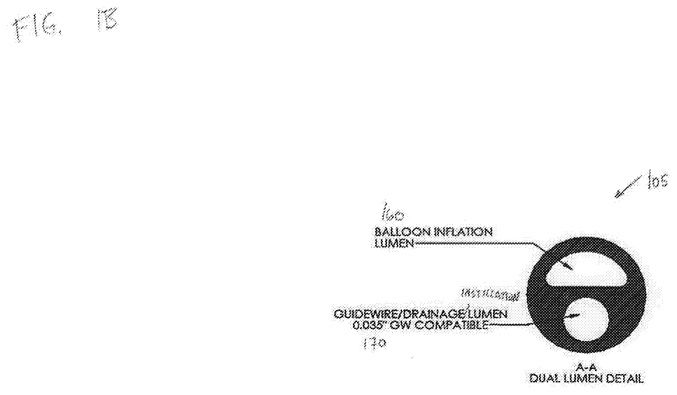

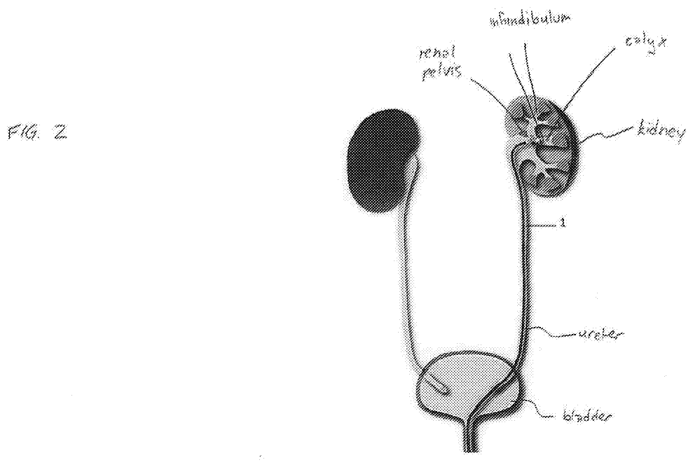

The invention relates to systems and methods for administering a drug to a target site in a body lumen. One example of the invention relates to systems and methods for locally delivering a chemotherapeutic agent to an upper tract urothelial carcinoma (UTUC). FIG. 1A shows an example catheter 101 of the invention, which has a tube-like body with lumens passing through the body. The catheter 101 includes a hollow balloon inflation lumen 160 and a hollow guidewire/drainage/instillation lumen 170 extending from proximal end 111 of catheter 101, including proximal end of balloon inflation lumen 161 and proximal end of guidewire/drainage/instillation lumen 171, respectively. The proximal end of the balloon inflation lumen 161 can function as a balloon infusion port in fluid communication with the balloon inflation lumen 160.

The balloon inflation lumen 160 and the guidewire/drainage/instillation (GDI) lumen 170 interface in Y-body Luer hub 165 to form two separate channels (lumens) in lumen shaft 105. The lumen shaft 105 is elastic and generally cylindrical and can be inserted into a luminal organ, including a ureter, a renal pelvis, an infundibulum, a renal calyx, and a kidney. As shown further in FIG. 1B, the lumen shaft 105 has a generally circular cross section and includes at least two lumens, balloon inflation lumen 160, and guidewire/drainage/instillation lumen 170. Balloon inflation lumen 160 and guidewire/drainage/instillation lumen 170 are formed longitudinally parallel through the length of lumen shaft 105. Lumen shaft 105 extends lengthwise from proximal ends of balloon inflation lumen and guidewire/drainage/instillation lumen 161, 171, along a working length WL to distal tip 150.

Catheter 101 includes an inflatable balloon 140 with an exposed outer surface 142 positioned near distal tip 150. The balloon 140 is in fluid communication with and receives a solution via balloon inflation lumen 160. In one example of the invention, balloon 140 is approximately 10-20 mm in length and is positioned 15 mm (from 10 mm to 20 mm, for example) from the end of distal tip 150. In its uninflated state (at rest), balloon 140 is approximately 8 mm in diameter and is coaxial with the center of lumen shaft 105. In its inflated state, balloon 140 remains 20 mm in length secured by RO marker bands 144 but can inflate to different diameters (up to approximately 20 mm) to occlude the different luminal organ of interest, including ureter, renal pelvis, and kidney. Example balloon volumes in their inflated state include balloon volumes of 2 cc to 12 cc. In one example of the invention, the balloon volume is 3 cc, and with 3 cc of fluid (e.g., water), the diameter of the balloon is approximately 1.6 cm. With 4 cc of fluid, the diameter of the balloon is approximately 1.8 cm (8 French and 24 French, respectively).

One example balloon catheter of the invention is made of silicone or PVC, for example, and has a diameter of about 3.2 French, an internal diameter of 0.38, and has a 1-cm elongated oval balloon. The balloon pressure can be adjusted as needed, and can, for instance, reach a maximum of 10 mm Hg. The catheter device can be inserted and positioned with assistance of an imaging technique, such as fluoroscopy, enhanced with a radiological contrast. In one example method of the invention, after positioning, the balloon is inflated to a volume sufficient to occlude the ureter and a liposomal formulation is instilled directly into the proximal ureter as described further below.

The balloon 140 is formed into a membrane form, from a heat-resistant resin or polymer such as polyurethane, PET (polyethylene terephthalate), elastomer, rubber, silicone, plastic, and other resins or polymers. In use, the balloon 140 is inflated to take the form of a rotating body, such as a substantially spherical form by filling a solution in the balloon 140. In one example of the invention, the solution is a mixture of physiological saline and a contrast agent, but the solution can be either a liquid or a gas, such as saline, sterile water, air, carbon dioxide, and other fluids. The shape of the balloon 140 can be other various shapes (other than the spherical shape) based upon the shape of an intraluminal wall, for example. For example, the balloon 140 can be a spherical shape with minor and major axes equal to each other, an oblate spheroid shape with a minor axis defined as a rotation axis, a prolate spheroid shape with a major axis defined as a rotation axis, a sand bag shape, and other shapes. Regardless of the specific shape of the balloon 140, the balloon 140 should be formed from an elastic member which is deformable when coming in close contact with an intraluminal wall.

Within the lumen shaft 105 of the catheter 101, balloon inflation lumen 160 provides a solution transport path communicating from a solution source (not shown separately) through Y-body Luer hub 165 to a filling portion formed inside the balloon 140 to deliver or convey the solution to the balloon 140 to inflate (and deflate) balloon 140. The solution source can include a chamber that communicates with the solution transport path (balloon inflation lumen 160) that holds a certain amount of the solution. A stopcock, syringe, valve, or other flow control device can be used to precisely regulate the amount of solution delivered from the solution source through the balloon inflation lumen 160 to balloon 140. As solution is delivered to the balloon 140, the balloon inflates. A piston, or other pressure regulating device, can be placed in line with the balloon inflation lumen 160 to keep balloon 140 inflated to a predetermined pressure or to a determined diameter to occlude the ureteral luminal organ of interest. To deflate the balloon 140, the pressure can be removed from the piston or other pressure regulating device, and the elasticity of the balloon 140 provides compression deformation to deflate the balloon 140 by forcing the solution back through the balloon inflation lumen 160 (from the distal end 112 of the catheter to the proximal end of the balloon inflation lumen 161) to the solution source.

In use, the inflation fluid remains in a closed system. When inflated, the inflation fluid only enters the balloon inflation lumen 160 and the interior of the balloon 140. When so inflated, the inflation fluid never exits the balloon inflation lumen 160 or the balloon 140 until a health care professional or other user specifically deflates the balloon 140, typically with a syringe or other flow control device similar to the one that was used to the inflate the balloon 140.

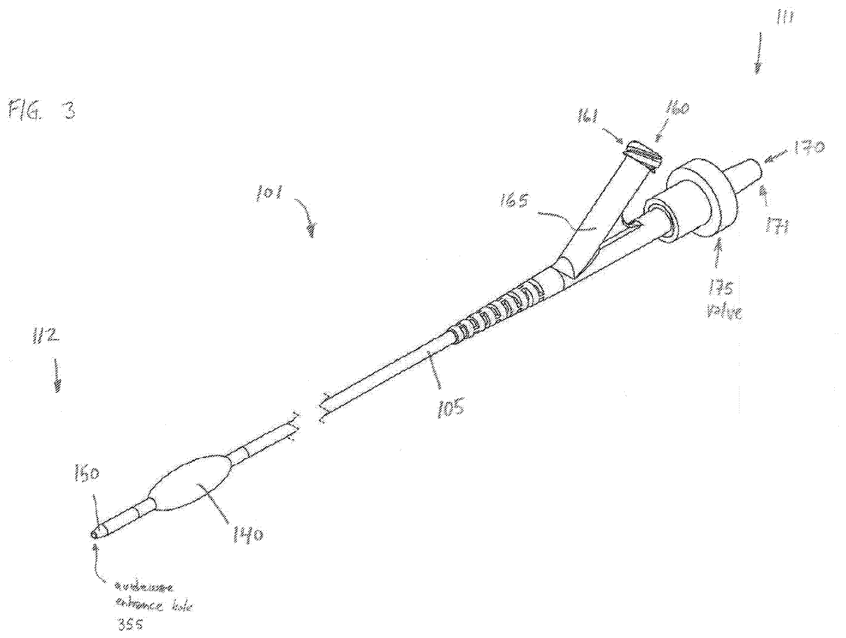

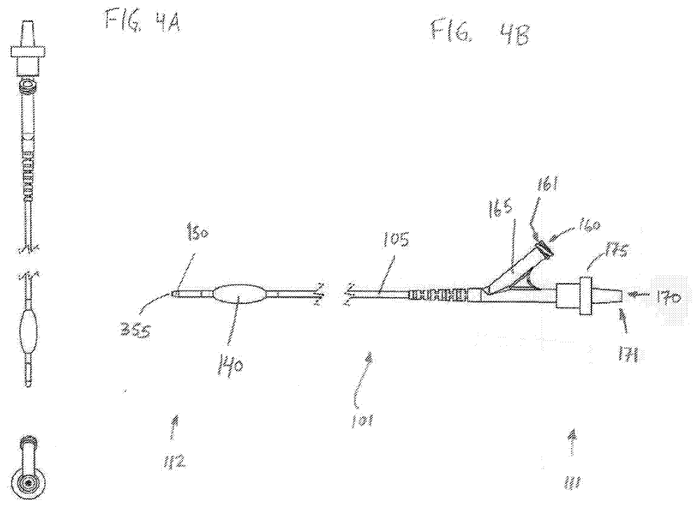

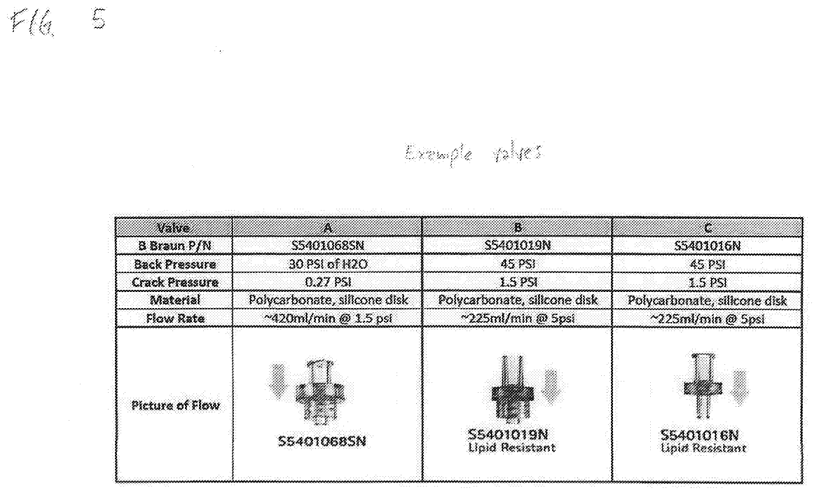

Guidewire/drainage/instillation lumen 170 receives a guidewire via guidewire entrance hole 355 (shown in FIG. 3), which guides distal tip 150 to a target site in a luminal organ target site. The guidewire/drainage/instillation lumen 170 is in fluid communication with the body cavity (e.g., the bladder, the ureter, the renal pelvis, or the kidney) for draining fluid from the body cavity. While this guidewire/drainage/instillation lumen 170 can be used for many purposes (e.g., carrying and delivering radiation therapy devices, instilling pharmaceutical products, placing a camera or other visualization device, removing an obstruction, and other uses), in the invention, the guidewire/drainage/instillation lumen 170 is used to infuse/instill a chemotherapeutic agent in the luminal organ, as described further below. The guidewire/drainage/instillation lumen 170 can also be used to instill radiographic contrast to define the anatomy of the upper ureteral collection system with fluoroscopy or radiography. The guidewire/drainage/instillation lumen 170 can also be used to collect a sample of urine for histological, cytological, or molecular assessment of urothelial cells.

Valve 175 is placed in-line with guidewire/drainage/instillation lumen 170 after the catheter 101 is placed in a treatment location. In one example of the invention, valve 175 is a modular attachment that mates to the catheter 110 and forms the proximal end of the guidewire/drainage/instillation lumen (GDI port) 171. With a modular construction, the valve 175 can be removed and/or replaced, such as through a conventional Luer or a screw-threaded connection.

Pyelorenal backflow is essentially a traumatic pressure phenomenon, for example, where leakage of the contrast medium injected up the ureter occurs at the calyceal fornix angle into the renal sinus fat and into the tributaries of the renal veins. To guard against this, some urologists, when injecting contrast media up the ureters, use a funnel and rubber tube and run it in by gravity. The pressure used in injecting with the hand syringe is surprisingly high, even up to 200 mm Hg. Pain is not a reliable guide to the state of filling of the renal pelvis during retrograde pyelography. Some patients tolerate pressures of over 100 mm Hg without discomfort, while some have severe discomfort at 20 mm. The appearance of backflow is a variable phenomenon, occurring at a pressure as low as 25 mm Hg, and pain may or may not accompany it.

The valve 175 is normally closed and can be set to provide pressure relieve to the guidewire/drainage/instillation lumen 170 when pressure in the luminal organ meets or exceeds a predetermined pressure, such as 15 mm Hg, for example. The pressure relief setting of the valve 175 will be lower than the maximum ureteral pressure, which can be measured or estimated prior to using the catheter 101. The valve 175 of the invention can be set to permit release of luminal fluid/contents into the guidewire/drainage/instillation lumen 170 when the predetermined pressure is exceeded in the luminal organ or in the guidewire/drainage/instillation lumen 170. As shown in FIGS. 1A and 3, the valve 175 can be located near the proximal end of guidewire/drainage lumen 171, for example. As pressure builds up in the luminal organ from urine production and collection while balloon 140 occludes the luminal organ, the pre-determined pressure is exceeded and valve 175 opens to allow urine to drain from the distal tip 150 of the catheter 101 toward the proximal end of the guidewire/drainage/instillation lumen 171. Removing a volume of urine from the luminal organ relieves pressure (i.e., pressure decreases as intraluminal volume decreases) in that organ and prevents injury and pyelovenous exposure to the patient. When the pressure in the luminal organ drops to below the predetermined pressure, the valve 175 closes again and remains closed unless the pressure again builds to meet or exceed the predetermined pressure, when the valve 175 opens again. The normally-closed one-way check valve provides for intermittent injection of fluids while preventing back flow. The valve opens automatically when pressure is applied.

The valve 175 includes construction components that do not permit fluid from exiting the lumen 170 until a given pressure is overcome. For example, the valve 175 can include a housing with a lumen, a ball, and a spring within the lumen where the spring presses the ball against a defined opening. When pressure on the ball exceeds the force of the spring, the ball moves away from the defined opening and fluid moves around the ball and vents to the proximal end 171. By controlling tension on the spring, the pressure at which the valve releases pressure can be controlled. It can also be appreciated that the pressure release valve can be coupled to a Luer connector, which can be further coupled to additional valves, collection sources, and other drainage apparatus. The valve 175 can be selected based on pressure control characteristics. For example, pyelovenous feedback occurs around 15-20 cm of water pressure (or about 0.27 psi.), so our crack pressure (that is, the pressure at which the valve releases the fluid) should be around 0.27 PSI. FIG. 5 shows a number of example valves and their performance characteristics.

This given pressure can be selectable by the physician depending upon the one-way valve that is chosen for use, where the invention includes a set of one-way valves having different predetermined pressures that are available for use by the physician. As outlined above, the pressure can be set to correspond to desired maximum ureteral pressure values. Therefore, when used, the fluid exits the valve 175 at the proximal end of the guidewire/drainage/instillation lumen 171 well before the patient's maximum ureteral pressure is exceeded.

The methods involve placing a balloon catheter 101 that has a working channel (guidewire/drainage/instillation lumen 170) and a balloon 140 into the ureter/renal pelvis via retrograde (or antegrade) upper urothelial tract access. The catheter balloon 140 is inflated to temporarily obstruct the ureter, and a liquid liposomal formulation with a chemotherapeutic agent (or a non-liposomal chemotherapeutic formulation) is infused/instilled into the working channel (guidewire/drainage/instillation lumen 170) of the catheter 101. The infused liposomal formulation is allowed to dwell in the ureter and/or renal pelvis for a period of time sufficient to allow at least a portion of the formulation to adhere to (and partially penetrate) the urothelial wall. In methods of the invention, the dwell time is from about 1 to about 120 minutes. For example, the liposomal formulation can be allowed to dwell for at least 1 minute, at least 10 minutes, at least 15 minutes, at least 20 minutes, at least 30 minutes, at least 45 minutes, at least 60 minutes, at least 75 minutes, at least 90 minutes, at least 105 minutes, or at least 120 minutes. In the methods of the invention, the infused chemotherapeutic-agent contacts and adheres, to the urothelial wall while it is instilled and dwells in the ureter and/or renal pelvis.

As indicated above, the invention can be used to deliver a therapeutic dose of a drug to the ureter and/or renal pelvis via instillation. More particularly, the invention is generally used to deliver a chemotherapeutic drug formulation, such as, but not limited to, a taxane class drug, a platinum-based drug, 5-fluorouracil, mitomycin C, gemcitabine, epirubacin, or thiotepa, for the purpose of treating UTUC. Preferred chemotherapeutic drug formulations are liposomal formulations of chemotherapeutic drugs. Liposomal formulations according to the invention, generally, contain at least one phospholipid component that is a pharmaceutically acceptable phospholipid or mixture of pharmaceutically acceptable phospholipids. Natural as well as synthetic phospholipids may be used. Examples of phospholipids in liposomal formulations used with the invention include, but are not limited to distearoyl phosphatidylcholine, dipalmitoyl phosphatidylcholine, dimyristoyl phosphatidylcholine, egg phosphatidylcholine, soy phosphatidylcholine, dimyristyl phosphatidyl glycerol sodium, 1,2-dimyristoyl-phosphatidic acid, dipalmitoylphosphatidylglycerol, dipalmitoyl phosphate, 1,2-distearoyl-sn-glycero-3-phospho-rac-glycerol, 1,2-distearoyl-sn-glycero-3-phosphatidic acid, phosphatidylserine and sphingomyelin. For example a liposomal formulation used with the invention may contain dimyristoyl phosphatidylcholine (DMPC) and dimyristoyl phosphatidyl glycerol sodium (DMPG). Therefore, a liposomal formulation delivered by a catheter described herein may contain paclitaxel, DMPC and DMPG. For example, the respective weight/weight ratios of paclitaxel, DMPC and DMPG in a liposome used with the invention may be 1:(1-10):(1-10), or any ratios therein.

A liposomal formulation according to the invention may also be composed of, or partially composed of lipids other than phospholipids, such as lipids containing no phosphoric acid, like a glycerolipid or a sphingolipid. Alternatively, liposomal formulations used with the invention, including liposomal formulations that contain one or more lipids other than phospholipids, may additionally contain a cholesterol, or cholesterol derivative component.

Liposomal formulations that can used with the invention also include formulations disclosed in: WO 2019/018619, which is directed to liposome formulations that include paclitaxel, lecithin, cholesterol, threonine, and glucose, including the paclitaxel formulation marketed under the trade name, Lipusu; and U.S. Patent Publication No. 2019/0015334 (“USPPN 2019/0015334”), which discloses liposomal formulations of paclitaxel, docetaxel, or cisplatin.

More preferred formulations for use with the invention are those described in USPPN 2019/0015334, which contain either paclitaxel, docetaxel, or cisplatin in combination with dimyristoyl phosphatidylcholine (DMPC) and dimyristoyl phosphatidyl glycerol sodium (DMPG). The most preferred formulations in USPPN 2019/0015334 contain (A) drug (paclitaxel, docetaxel, or cisplatin), (B) DMPC; and (C) DMPG in weight/weight ratios of (A):(B):(C) of (1):(1.3-4.5):(0.4-2.5), or any ratios therein, such as the combination of paclitaxel, DMPC and DMPG w/w ratios of (1):(1.43):(0.47).

A therapeutic dose of a chemotherapeutic drug is an amount of drug, that, when delivered to the ureter or renal pelvis of a subject with UTUC, ameliorates a sign or symptom of UTUC. For example, a therapeutic dose of a drug, when administered by the invention in either a single administration or multiple administrations, causes a reduction in tumor burden or a decrease in the number of size of metastases. The terms “ameliorating” or “treating” refer to the reduction in the number or severity of signs or symptoms of a disease, including the complete eradication of the disease. The amount of drug in a therapeutic dose, which may also be called an “effective dose”, depends on the stage, severity and course of the UTUC, previous therapy, the individual's health status, weight, response to the drugs, and/or the judgment of the treating physician. Exemplary therapeutic dose for a method of the invention include an amount of paclitaxel that ranges from is 1 to 1000 mg/m². For example, the therapeutic dosage may be from: 1 to 100 mg/m²; 50 to 150 mg/m²; 100 to 200 mg/m²; 150 to 250 mg/m²; 200 to 300 mg/m²; 350 to 450 mg/m²; 400 to 500 mg/m²; 450 to 550 mg/m²; 500 to 600 mg/m²; 550 to 650 mg/m²; 600 to 700 mg/m²; 750 to 850 mg/m²; 800 to 900 mg/m²; 850 to 950 mg/m²; 900 to 1000 mg/m²; or 950-1000 mg/m². The concentration of the administered therapeutic dose of paclitaxel in an exemplary use of the invention—the amount of paclitaxel instilled by a catheter according to the invention to the ureter or renal pelvis—may, for example, be at least 0.5 mg/ml, at least 1 mg/ml, at least 1.5 mg/ml, at least 2 mg/ml, at least 2.5 mg/ml, at least 3 mg/ml, at least 3.5 mg/ml, at least 4 mg/ml, at least 4.5 mg/ml, at least 5 mg/ml, or any concentration therein.

What is claimed is:

1. A catheter 101 having a proximal end 111 and a distal end 112, the catheter 101 comprising:
a balloon inflation lumen 130 in fluid communication with a balloon infusion port 161 in the proximal end 111 of the catheter 101 and also in fluid communication with an expandable occlusion balloon 140 in the distal end 112 of the catheter 101;
a guidewire/drainage/instillation lumen 170 in fluid communication with a guidewire/drainage/instillation port 171 in the proximal end 111 of the catheter 101 and also in fluid communication with at least one hole in the distal end 112 of the catheter 101, the guidewire/drainage/instillation lumen 170 for passing urine from the at least one hole to the guidewire/drainage/instillation port 171; and
a guidewire entrance hole 355 in fluid communication with the guidewire/drainage/instillation lumen 170 and located in the distal end 112 of the catheter 101, the guidewire entrance hole 355 to load and advance the catheter 101 over a guidewire with the guidewire passing through the guidewire entrance hole 355, the guidewire/drainage/instillation lumen 170, and the guidewire/drainage/instillation port 171;
a pressure relief valve 175 disposed along the guidewire/drainage/instillation lumen 170 between the guidewire/drainage/instillation port 171 and the expandable occlusion balloon 140 and configured to release luminal fluid into the guidewire/drainage/instillation lumen 170 when a predetermined ureteral pressure is exceeded; and
the expandable occlusion balloon 140 in fluid communication with the balloon inflation lumen 160 and disposed at the distal end 112 of the catheter 101, wherein the expandable occlusion balloon 140 is configured to withstand a force greater than the predetermined ureteral release pressure of the valve 175.

2. The catheter 101 of claim 1, wherein the expandable occlusion balloon 140 is configured to be expanded within a ureter of a patient.

3. The catheter 101 of claim 1, wherein the expandable occlusion balloon 140 is configured to be expanded within a ureteral lumen of a patient.

4. The catheter 101 of claim 1, wherein the expandable occlusion balloon 140 is an occlusion balloon.

5. The catheter 101 of claim 1, wherein the expandable occlusion balloon 140 is a dilation balloon.

6. The catheter 101 of claim 1, wherein at least one of the balloon infusion port 161 and the guidewire/drainage/instillation port 171 is detachable from the catheter 101.

7. A method for treating upper tract urothelial carcinoma (UTUC) in a subject in need thereof, comprising delivering a chemotherapeutic agent to the ureter or renal pelvis of the subject, the method comprising:
A. placing a catheter according to claim 1 into the ureter;
B. expanding the occlusion balloon 140 balloon of the catheter such that the ureter or renal pelvis is temporarily obstructed;
C. loading a formulation comprising a therapeutic dose of a chemotherapeutic drug into the guidewire/drainage/instillation lumen 170 of the catheter;
D. instilling the formulation into the ureter or renal pelvis; and
E. allowing the instilled formulation to dwell in the ureter or renal pelvis for a time sufficient to allow at least a portion of the formulation to adhere to and partially penetrate the urothelial wall.

8. The method according to claim 7, wherein the chemotherapeutic agent is at least one of a taxane class drug, a platinum-based drug, 5-fluorouracil, mitomycin C, gemcitabine, epirubacin, thiotepa, or a combination thereof.

9. The method of claim 8, wherein the formulation is a liposomal formulation.

10. The method of claim 9, wherein the liposomal formulation comprises:
(A) a taxane class drug or a platinum-based drug;
(B) dimyristoyl phosphatidylcholine (DMPC), and
(C) dimyristoyl phosphatidyl glycerol sodium (DMPG) wherein the weight/weight ratios of A:B:C are 1:(1.3-4.5):(0.4-2.5)

11. The method of claim 10, wherein the taxane class drug is paclitaxel, and the weight/weight ratios of A:B:C are (1):(1.43):(0.47).

12. A method according to claim 7, wherein the instilled formulation is allowed to dwell in the at least one of the ureter and renal pelvis for at least about 1 minute, about 10 minutes, about 15 minutes, about 20 minutes, about 30 minutes, about 45 minutes, about one hour, or about 2 hours.

13. A method for visualizing a ureter or renal pelvis of a subject, the method comprising:
A. placing a catheter according to claim 1 into the ureter;
B. expanding the occlusion balloon 140 balloon of the catheter such that the ureter or renal pelvis is temporarily obstructed;
C. loading a composition comprising a radiographic contrast agent into the guidewire/drainage/instillation lumen 170 of the catheter;
D. instilling the composition comprising the radiographic contrast agent into the ureter or renal pelvis; and
E. allowing the instilled composition to dwell in the ureter or renal pelvis for a time sufficient to allow to allow visualization of the ureter or renal pelvis with fluoroscopy or radiography, while minimizing pyelo-venous backflow exposure to the patient.
